(19) United States
(12) Patent Application Publication
(10) Pub. No.: US 2026/0185993 A1
(43) Pub. Date: Jul. 2, 2026

(54) FUNCTIONAL PEPTIDE MODIFIED MAGNETIC COMPOSITE NANOBEAD AND PREPARATION METHOD THEREOF

(71) Applicant: Institute of Sensing Technology, Gansu Academy of Sciences, Lanzhou (CN)

(72) Inventors: Xianwei ZUO, Lanzhou (CN); Yan WANG, Lanzhou (CN); Zhiqi FENG, Lanzhou (CN); Chunyang KANG, Lanzhou (CN); Chunyan LIU, Lanzhou (CN); Xiaoping GAO, Lanzhou (CN); Yidan LIU, Lanzhou (CN); Xin LI, Lanzhou (CN); Xin HE, Lanzhou (CN)

(21) Appl. No.: 19/263,583

(22) Filed: Jul. 9, 2025

(30) Foreign Application Priority Data
Dec. 31, 2024 (CN) 202411989760.X

(51) Int. Cl. G01N 33/573; G01N 33/533; G01N 33/543

(52) U.S. Cl. CPC G01N 33/573; G01N 33/533; G01N 33/54326; G01N 2333/988; G01N 2500/04

(57) ABSTRACT

A functional peptide modified magnetic composite nanobead, and a method and an application thereof for detecting β-secretase (BACE1) and screening its inhibitors are provided. The chemical composition of the magnetic composite nanobead is: ferroferric oxide (Fe₃O₄) nanoclusters used as magnetic cores, the surface of the magnetic cores coated with a silica shell, the surface of the shell connected with an ethylene diamine tetraacetic acid (EDTA) modification layer, and Co²⁺ or Ni²⁺ ions chelated on the EDTA layer, and the Cy5 fluorescein labeled functional peptide coated on the surface of magnetic composite nanobead by the interaction between Co²⁺ or Ni²⁺ ions and hexahistidine-tag.

### CROSS-REFERENCE TO RELATED APPLICATIONS

This application claims priority to Chinese Patent Application No. 202411989760.X, filed on Dec. 31, 2024, the contents of which are hereby incorporated by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: SequenceListing.xml
Creation date: Jul. 8, 2025
Byte size: 4,918

### TECHNICAL FIELD

The disclosure belongs to the technical field of nanomaterial preparation and detection, and more particular to a functional peptide modified magnetic composite nanobead, a preparation method and an application for detecting β-secretase and screening its inhibitors thereof.

### BACKGROUND

With the acceleration of global population aging, Alzheimer's disease (AD), which has a high incidence and severe consequences in the elderly population, has become a serious threat to public health. A transmembrane aspartic protease, β-secretase (BACE1), is known to be a key enzyme in the processing of amyloid precursor protein generates toxic amyloid-O peptide which may cause Alzheimer's disease, so it has always been regarded as an important target for treating AD. In addition, BACE1 also plays an important role in abnormal metabolism, vascular function and immune function. Abnormal increase of BACE1 may directly or indirectly accelerate the aging of a body and promote development of aging-related diseases (such as cardiovascular and cerebrovascular diseases, diabetes and cancers). Therefore, it is of great significance to detect BACE1 activity and screen its inhibitors in a biomedical field.

Magnetic nanomaterials are a class of multi-performance nanomaterials, which not only have characteristics of nanomaterials, such as small particle size and large specific surface area, but also have special magnetic orientation, superparamagnetism and relaxation properties. Furthermore, magnetic nanomaterials may gather and locate under an action of external magnetic field, so they may realize rapid separation-dispersion cycle operation and may be reused. These unique optical properties make magnetic nanomaterials as ideal nanoprobes for detection of biomolecules.

To use magnetic nanomaterials as nano-bioprobes for detecting proteases, the surface of magnetic nanomaterials should be modified with the peptide substrate. So far, in many researches the most popular modified method is to form amide bonds between carboxyl groups (introduced to the surface of magnetic nanomaterials) and primary amines of peptide substrate. However, there is a possibility that the enzyme recognition site of the peptide substrate is blocked by the non-selective formation of amide bonds. To overcome these challenges, much effort is needed to explore new and facile methods for preparing peptide substrate modified magnetic nanomaterials to detect BACE1.

Based on above discussion, a novel functional peptide modified magnetic composite nanobead and its preparation method are provided in the present disclosure, and the magnetic composite nanobead as nano-bioprobe may sensitively detect BACE1 and screen its inhibitors, which has potential application value in the field of AD drug development.

### SUMMARY

In view of the above technical problems, the first object of the present disclosure is to provide a functional peptide modified magnetic composite nanobead, and a chemical composition of the functional peptide modified magnetic composite nanobead is as follows: ferroferric oxide (Fe₃O₄) nanoclusters are used as magnetic cores, the surface of the magnetic cores is coated with a silica shell, the surface of the silica shell is connected with an ethylene diamine tetraacetic acid (EDTA) modification layer, and Co²⁺ or Ni²⁺ ions are chelated on the EDTA layer, and Cy5 fluorescein labeled functional peptide is coated on the surface of the magnetic composite nanobead by the interaction between Co²⁺ or Ni²⁺ ions and hexahistidine-tag; and an amino acid sequence of the Cy5 fluorescein labeled peptide is as follows:

(SEQ ID No. 1)
(1)Cys-Gly-Gly-Ser-Ser-Glu-Val-Asn-Leu-
Asp-Ala-Glu-Phe-Ser-Ser-Gly-Leu-His-
His-His-His-His-His;
(SEQ ID NO. 2)
(2)Lys-Thr-Glu-Glu-Ile-Ser-Glu-Val-Asn-
Leu-Asp-Ala-Glu-Phe-Arg-His-Asp-Lys-
Cys-His-His-His-His-His-His;
(SEQ ID No. 3)
(3)Cys-Leu-Gly-Gly-Glu-Val-Asn-Leu-Asp-
Ala-Glu-Phe-Gly-Gly-Leu-His-His-
His-His-His-His;
(SEQ ID No. 4)
(4)[(His-His-His-His-His-His)4Lys]-
Gly-Gly-Pro-Pro-Pro-Pro-Pro-Pro-Pro-Pro-
Pro-Gly-Glu-Val-Asn-Leu-Asp-Ala-Glu-Phe-
Asp-Asp-Lys-Gly-Gly-Asp-Asp-Asp-NH₂

as shown in any one of the (1), (2), (3), (4).

A second object of the present disclosure is to provide a method for preparing the functional peptide modified magnetic composite nanobead, including the following steps:
(1) Fe₃O₄ nanoclusters are prepared using a solvothermal method;
(2) the Fe₃O₄ nanoclusters as-prepared in the step (1) are taken as magnetic cores, and silica-coated Fe₃O₄ nanoclusters is prepared using an ultrasonic-assisted sol-gel method;
(3) the silica-coated Fe₃O₄ nanoclusters as-prepared in the step (2) are equably dispersed in methano by ultra-sonication, then glacial acetic acid and sodium N-(trimethoxysilyl propyl) ethylenediamine triacetate with the mass ratio of 1:2-1:10 are added into a mixed solution under a mechanical agitation, the mixed solution keep a reaction by ultra-sonication at 150-300 Watt (W) for 1-3 hours (h), the resulted materials are thoroughly washed with the deionized water and acetone, and then EDTA-modified silica-coated Fe₃O₄ nanoclusters are obtained;
(4) the EDTA-modified silica-coated Fe₃O₄ nanoclusters as-prepared in the step (3) are equably dispersed in deionized water, then aqueous solution of NiCl₂ or CoCl₂ is added in the mixed solution, in which a mass ratio of the EDTA-modified silica-coated Fe₃O₄ nanoclusters to NiCl₂ or CoCl₂ reached 1:10-1:100, the reaction mixture is further held under mechanical agitation for 1.5-3 h at 25 degrees Celsius (° C.), the resulted materials are centrifuged and washed with deionized water for three times, and Ni²⁺ or Co²⁺ chelated magnetic composite nanobeads are obtained for further experiments; and
(5) the Ni²⁺ or Co²⁺ chelated magnetic composite nanobeads as-prepared in the step (4) are dispersed in PBS buffer, then 5-15 micromole per liter (μmol/L) aqueous solution of functional peptides is added into the mixed solution, the reaction mixture is incubated at 37° C. for tens of minutes, the resulted materials are separated by external magnet and washed with PBS buffer for 3-5 times until a fluorescence intensity of a washing solution tends to zero, and the functional peptide modified nanobeads are obtained.

Optionally, specific steps of preparing Fe₃O₄ nanoclusters in the step (1) are as follows:
Ferric chloride and sodium acetate are completely dissolved in ethylene glycol, then a little deionized water is added to form a mixed solution, in which a mass ratio of ferric chloride, sodium acetate and deionized water is 1:3:1, the mixed solution is added into a reaction kettle, and heated to 140-170° C. for 1.5-3 h, then heated in a temperature range of 180-220° C. for 2-8 h, a whole reaction is maintained in a pressure range of 0.01-0.1 Pascal (Pa). After the reaction is completed, the obtained Fe₃O₄ nanoclusters are separated using external magnet and washed with acetone and anhydrous ethanol respectively.

Optionally, the method for preparing silica-coated Fe₃O₄ nanoclusters in the step (2) includes following specific steps.

A certain amount of Fe₃O₄ nanocluster particles as-prepared in the step (1) is equably dispersed in a mixed solvent of deionized water and anhydrous ethanol with a volume ratio of 1:5-4:5 by ultra-sonication, then, 25 percent (%) ammonia water and tetraethyl orthosilicate (TEOS) with a volume ratio of 1:10-1:20 are added into the mixed solution, then ultrasonic reaction of the mixture is carried out for 20-60 minutes (min) in a power of 180-360 W under an ice water bath environment, after the reaction is completed, the obtained silica-coated Fe₃O₄ nanoclusters are separated using external magnet, and washed with deionized water and anhydrous ethanol respectively.

A third object of the present disclosure is to provide a method for detecting (3-secretase (BACE1) by using the functional peptide modified magnetic composite nanobead for non-diagnostic purposes, including the following steps:
A certain amount of functional peptide modified magnetic composite nanobead are equably dispersed in acetic acid-acetate (HAc-NaAc) buffer, and then different concentrations of target β-secretase are added into the reaction buffer, and the mixture reaction is performed at 37° C. for tens of minutes, and after magnetic composite nanobeads are separated using external magnet, the supernatants of reaction solution are collected, and the fluorescence intensity of obtained solutions is tested for fitting the regression equation of β-secretase, so the standard curve for BACE1 detection may be established.

Optionally, the concentration of HAc-NaAc buffer is 20 millimole per liter (mmol/L), potential of hydrogen (pH) of the buffer is 4.5-6.0, a concentration of magnetic composite nanobead is 0.2-1.0 milligram per milliliter (mg/mL), and a reaction time is 60-120 min.

A fourth object of the present disclosure is to provide an application of the functional peptide modified magnetic composite nanobead in screening BACE1 inhibitors.

Compared with the prior art, the disclosure has the beneficial effects as follows.

The functional peptide modified magnetic composite nanobead provided by the present disclosure is prepared by Fe₃O₄ nanocluster, silica layer, EDTA modification layer, Co²⁺ or Ni²⁺ chelated layer, and Cy5 fluorescein and histidine labeled functional peptide through layer-by-layer assembly technology. It not only inherits the excellent properties of magnetic and fluorescent materials, such as magnetic separation and reuse, but also has a smaller particle size, larger specific surface area, higher magnetic response and more biomolecule loading than commercially available magnetic composite nanobead. Moreover, because of directional coupling between magnetic nanomaterials and peptide chains, functional peptides may effectively expose a BACE1 cleavage site, and exhibit outstanding ability to recognize and detect BACE1. Therefore, functional peptide modified magnetic composite nanobeads have multiple excellent performance.

The method for preparing the functional peptide modified magnetic composite nanobead provided by the present disclosure may be easily expanded to prepare other biomolecule modified magnetic composite nanobeads by changing His tagged peptide or protein, which has a general applicability and designability. In addition, preparation condition of the magnetic composite nanobead is simple and mild, which is beneficial to a large-scale preparation.

The method for detecting BACE1 using functional peptide modified magnetic composite nanobead has a detection linear range of 2.5 nanogram per milliliter (ng/mL) to 800 ng/mL and a detection limit of 0.62 ng/mL. This method has the advantages of high sensitivity, strong anti-interference ability, small background signal and magnetic separation and reuse.

### BRIEF DESCRIPTION OF THE DRAWINGS

FIG. 1 is a flow chart of preparing functional peptide modified magnetic composite nanobead in Embodiment 1.

FIG. 2 is a flow chart of preparing functional peptide modified magnetic composite nanobead.

FIG. 3 is a flow chart of preparing Fe₃O₄ nanoclusters by solvothermal method.

FIG. 4 is a flow chart of preparing silica-coated Fe₃O₄ nanocluster particles by the ultrasonic-assisted sol-gel method.

FIG. 5A is a transmission electron microscope (TEM) image of silica-coated Fe₃O₄ nanoclusters prepared in Embodiment 1.

FIG. 5B is an enlarged TEM image of Fe₃O₄NCs@SiO₂ nanoparticles prepared in Embodiment 1.

FIG. 6 is a Fourier transform infrared spectoscopy (FT-IR) pattern of EDTA-modified magnetic nanoparticles prepared in Embodiment 1.

FIG. 7A is an X-ray photoelectron spectroscopy (XPS) full spectrum of a Ni²⁺ chelated magnetic nanoparticles prepared in Embodiment 1.

FIG. 7B is a Ni 2p photoemission spectrum of nanoparticles prepared in Embodiment 1.

FIG. 8 is a magnetic hysteresis loop (VSM) diagram of Ni²⁺ chelated magnetic nanoparticles prepared in Embodiment 1.

FIG. 9 is a fluorescence microscope diagram of the functional peptide modified magnetic composite nanobead prepared in Embodiment 1.

FIG. 10 is a TEM image of Fe₃O₄NCs@SiO₂ nanoparticles prepared in Embodiment 2.

FIG. 11 shows the effect of different buffer systems when Fe₃O₄NCs@SiO₂ is connected with EDTA.

FIG. 12A is the XPS full spectrum of Co²⁺ chelated nanoparticles prepared in Embodiment 4.

FIG. 12B is a Co 2p photoemission spectrum of magnetic nanoparticles prepared in Embodiment 4.

FIG. 13 shows a fluorescence spectrum of magnetic composite nanobead prepared by different fluoresceins labeled functional peptides.

FIG. 14 is a comparative diagram of fluorescence intensity when different peptide chains modified magnetic composite nanobead is used for detecting BACE1 under the same conditions.

FIG. 15 shows the potential of hydrogen (pH) effect of buffer systems on fluorescence intensity for BACE1 detection.

FIG. 16 shows fluorescence emission spectra upon addition of various concentrations of BACE1.

FIG. 17 shows the relationship between fluorescence intensity and concentration of BACE1.

FIG. 18 shows the selectivity for BACE1 detection using functional peptide modified magnetic composite nanobead.

FIG. 19 is a dose response curve generated from screening assay of BACE1 inhibitors using functional peptide modified magnetic composite nanobead.

### DETAILED DESCRIPTION OF THE EMBODIMENTS

In order to make the technical means, creative features, goals and effects of the disclosure easy to understand, the disclosure will be further elaborated with specific embodiments. The following embodiments are only used to illustrate the technical scheme of the present disclosure, but not to limit it. Although the present disclosure has been described in detail with reference to preferred embodiments, it should be understood by those skilled in the art that the technical scheme of the present disclosure may be modified or replaced by equivalents without departing from the purpose and scope of the technical scheme, which should be included in the scope of the claims of the present disclosure.

In the following embodiments, the used reagents and consumables are all commercially available.

### Embodiment 1 Preparation of Functional Peptide Modified Magnetic Composite Nanobead 1

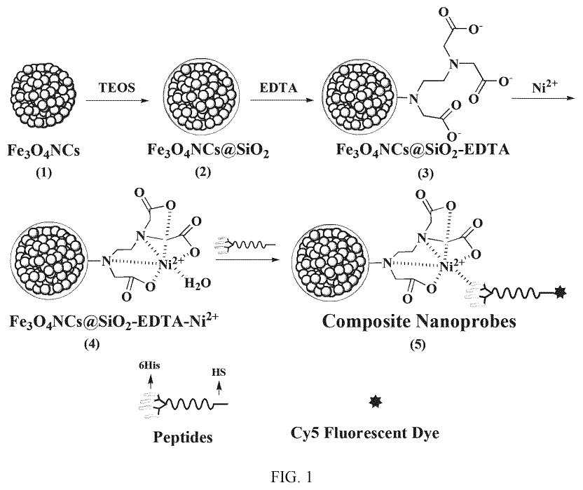

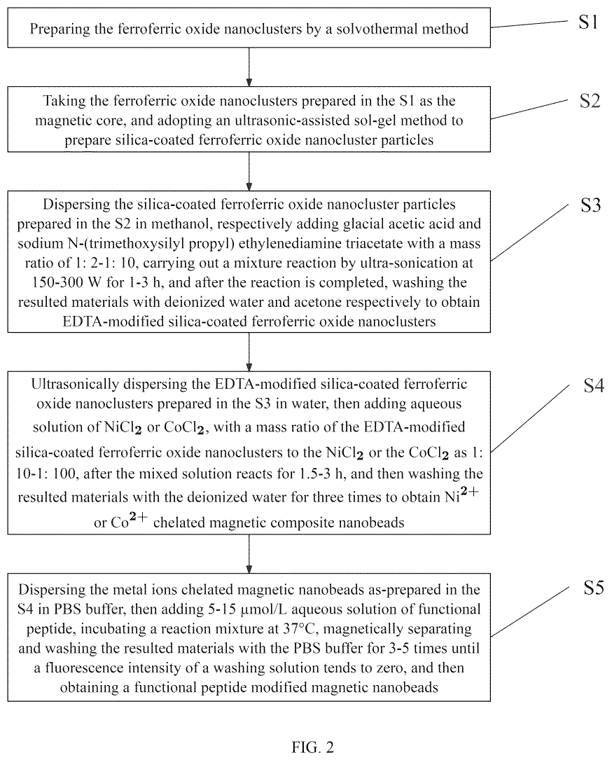

The preparation process of functional peptide modified magnetic composite nanobead is shown in FIG. 1 and FIG. 2, and the specific steps are as follows.

(1) Preparation of Fe₃O₄ Nanoclusters (Fe₃O₄NCs)

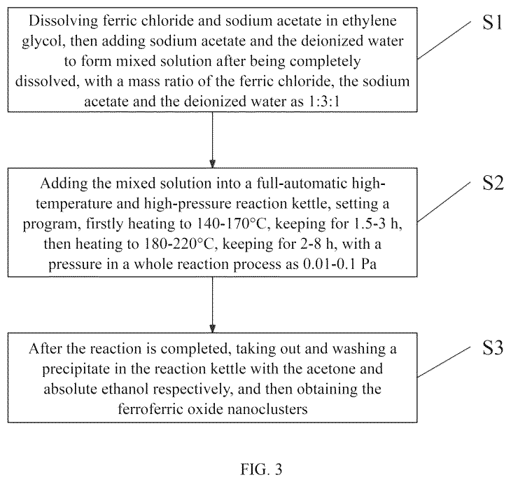

As shown in FIG. 3, 0.34 gram (g) of FeCl₃·6H₂O and 0.9 g of NaAC·3H₂O are completely dissolved in 60 milliliter (mL) ethylene glycol, then 0.3 mL of deionized water is added to form mixed solution. Subsequently, the mixed solution is added to a reaction kettle, and heated at 160 degree Celsius (° C.) for 3 hours (h), and then heated at 200° C. for 8 h. The whole reaction is maintained in a pressure range of 0.01-0.1 Pascal (Pa). After the reaction is completed, the obtained Fe₃O₄ nanoclusters are separated using external magnet and washed with acetone and anhydrous ethanol respectively.

(2) Preparation of Silica-Coated Magnetic Bead (Fe₃O₄NCs@SiO₂)

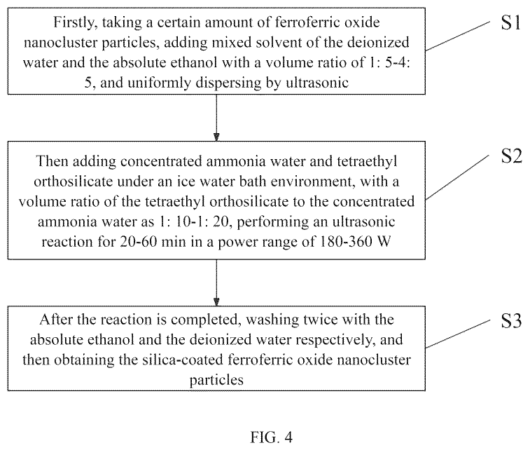

As shown in FIG. 4, 35 milligram (mg) of Fe₃O₄ nanoclusters are ultrasonically dispersed in a mixed solvent containing 12 mL of deionized water and 20 mL of anhydrous ethanol. Subsequently, 2 mL of concentrated ammonia water and 100 microliter (μL) of tetraethyl orthosilicate (TEOS) are added into the mixed solution, and then the ultrasonic reaction of the mixture is carried out at 220 Watt (W) for 40 minutes (min) under an ice water bath environment. After the reaction is completed, the obtained silica-coated Fe₃O₄ nanoclusters (Fe₃O₄NCs@SiO₂) are separated using external magnet and washed with deionized water and anhydrous ethanol respectively.

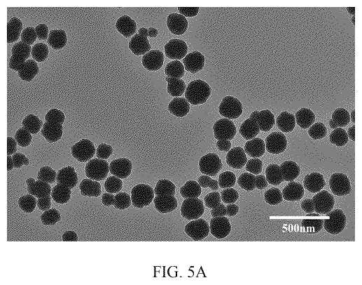

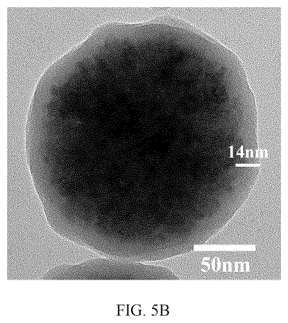

TEM image of the obtained Fe₃O₄NCs@SiO₂ nanoparticles is shown in FIG. 5A and the enlarged TEM image of a nanoparticle is shown in FIG. 5B. As observed in the images, the magnetic nanoparticles exhibit a core-shell structure with a shell thickness of about 14 nanometer (nm) and its average particle size is approximately 160 nm. The magnetic core, composed of Fe₃O₄NCs, consists of tiny Fe₃O₄ nanoclusters assembled into packed spherical particles. The Fe₃O₄NCs@SiO₂ magnetic nanoparticles demonstrate excellent dispersion and uniform size distribution.

(3) Preparation of EDTA-Modified Magnetic Bead (Fe₃O₄NCs@SiO₂-EDTA)

200 mg of Silica-coated Fe₃O₄ nanoclusters are equably dispersed in 50 mL of methanol by ultra-sonication, and then 50 μL of glacial acetic acid and 400 μL of sodium N-(trimethoxysilyl propyl) ethylenediamine triacetate are added into the mixed solution under a mechanical agitation. The mixed solution keeps a reaction by ultra-sonication at 300 W for 2 h. After that, the resulted materials are thoroughly washed with the deionized water and acetone, and then EDTA-modified silica-coated Fe₃O₄ nanoclusters are obtained.

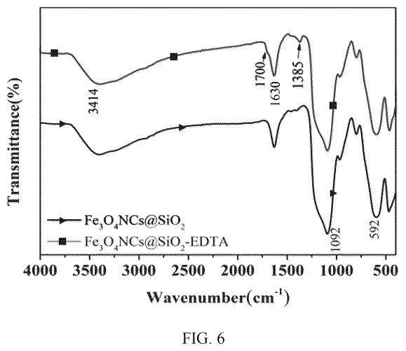

The FT-IR spectra of Fe₃O₄NCs@SiO₂-EDTA and Fe₃O₄NCs@SiO₂ are shown in FIG. 6. As observed in the spectra, a distinct peak appears at approximately 1385 reciprocal centimeter (cm⁻¹), corresponding to the symmetric stretching vibration of carboxylate groups. In addition, a weak but discernible absorption band observed at 1700 cm⁻¹ may be attributed to the C═O stretching vibration of protonated carboxylic acid (—COOH) groups, consistent with hydrogen-bonded dimer formation in the solid state. The result confirms the successful conjugation of EDTA onto the surface of Fe₃O₄NCs@SiO₂. In order to measure the carboxyl content of magnetic beads, a conductometric titration test is carried out, and the carboxyl content of Fe₃O₄NCs@SiO₂-EDTA magnetic composite nanobead is calculated to 320.3 micromole per gram (μmoL/g).

(4) Preparation of Ni²⁺ Ion Chelating Magnetic Nanoparticles (Fe₃O₄NCs@SiO₂-EDTA-Ni²⁺)

300 μL of 20 milligram per milliliter (mg/mL) Fe₃O₄NCs@SiO₂-EDTA water dispersion are taken in a 5 mL EP tube, then 4 mL of 1 molar concentration per liter (mol/L) NiCl₂ solution is added, and the reaction mixture is further held under mechanical agitation for 2 h; after, the resulted materials are centrifuged and washed with deionized water for three times, and then Ni²⁺ chelated magnetic composite nanobeads are obtained.

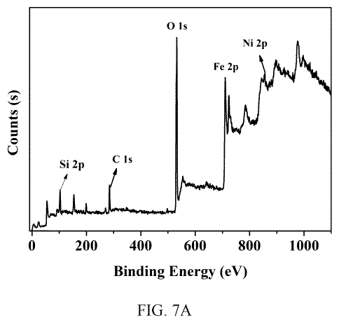

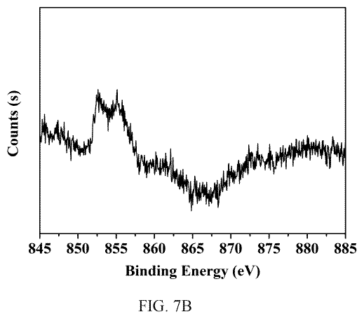

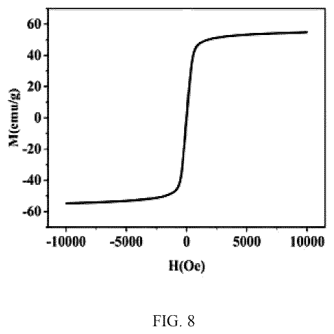

The XPS survey scan of Fe₃O₄NCs@SiO₂-EDTA-Ni²⁺ (FIG. 7A) exhibits four characteristic peaks at binding energies of 101.8 electron volt (eV) (Si 2p), 284.8 eV (C 1s), 531.6 eV (O 1s), and 712.5 eV (Fe 2p). Additionally, as shown in FIG. 7B, a distinct Ni²⁺ 2p photoelectron peak is observed at 855.7 eV, which confirms that Ni²⁺ ions are chelated onto the surface of Fe₃O₄NCs@SiO₂-EDTA magnetic beads. The magnetic properties of Fe₃O₄NCs@SiO₂-EDTA-Ni²⁺ magnetic beads are also tested using a vibrating sample magnetometer (VSM). As shown in the room-temperature hysteresis loop in FIG. 8, the saturation magnetization reaches 57 electromagnetic unit per gram (emu/g) with coercivity close to zero, indicating superparamagnetic behavior.

### (5) Preparation of Functional Peptide Modified Magnetic Composite Nanobead

A Cy5-labeled functional peptide is designed and synthesized by the biotechnology company, which comprises amino acid sequence as follows: [(His-His-His-His-His-His)4Lys]-Gly-Gly-Pro-Pro-Pro-Pro-Pro-Pro-Pro-Pro-Pro-Gly-Glu-Val-Asn-Leu-Asp-Ala-Glu-Phe-Asp-Asp-Lys-(Cy5)-Gly-Gly-Asp-Asp-Asp-NH₂, and then is prepared into a 100 micromolar per liter (mol/L) aqueous solution for later use. Subsequently, Fe₃O₄NCs@SiO₂-EDTA-Ni²⁺ obtained in the step (4) are dispersed in 20 millimolar per liter (mmol/L) phosphate-buffered saline (PBS) (potential of hydrogen (pH)=6.0, with 0.05 percent (%) Tween-20, 300 mmol/L NaCl, and 10 mmol/L imidazole), and then 70 μL of 5 mg/mL magnetic composite nanobead suspension is transferred into an Eppendorf tube. Then 9 μL of 100 mol/L functional peptide solution in water is added into the mixed suspension, and the PBS solution is added in a final reaction volume of 600 μL. After that, the reaction mixture is incubated at 37° C. for 2 h. Finally, the resulted materials are separated by external magnet and washed with PBS buffer for 3-5 times until the fluorescence intensity of washing solution tends to zero, and then functional peptide modified nanobeads are obtained.

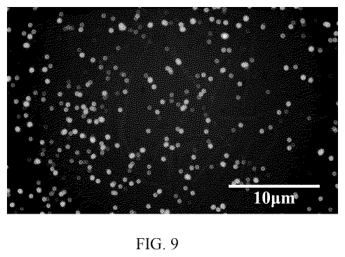

The fluorescence microscopy image of functional peptide modified magnetic composite nanobeads is shown in FIG. 9. It may be seen that magnetic composite nanobeads exhibited uniformly red fluorescent, which indicates Cy5-labeled functional peptides are successfully coated on the surface of magnetic composite nanobeads.

### Embodiment 2. Preparation of Functional Peptide Modified Magnetic Composite Nanobead 2

What is different from Embodiment 1 is:

In preparation step (1) of functional peptide modified magnetic composite nanobeads, the mass of FeCl₃·6H₂O and NaAC·3H₂O is respectively 0.68 g and 1.8 g, and 0.6 mL of deionized water is added; and in preparation step (2) of functional peptide modified magnetic composite nanobeads, 200 μL of TEOS is added into the mixed solution.

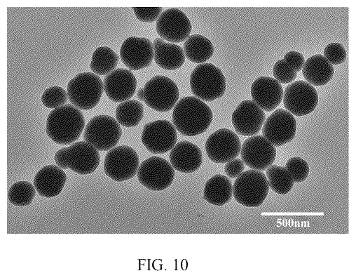

The TEM image of obtained magnetic composite nanobead in embodiment 2 is shown in FIG. 10. As may be seen, the average particle size of magnetic composite nanobead is about 350 nm, obviously larger than the average particle size of the magnetic composite nanobead prepared in Embodiment 1, and a particle size distribution is not uniform.

### Embodiment 3. Preparation of Functional Peptide Modified Magnetic Composite Nanobead 3

What is different from Embodiment 1 is:

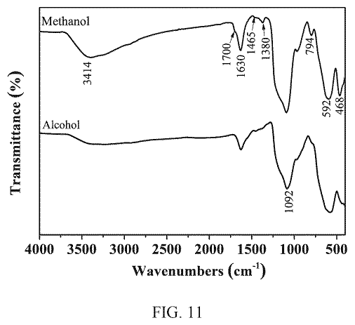

In preparation step (3) of functional peptide modified magnetic composite nanobeads, the alcohol is as the solvent, and other conditions remain unchanged. The FT-IR pattern of magnetic composite nanobeads is shown in FIG. 11, it may be seen, when the solvent of the reaction system is alcohol, there is no obvious peak at 1700 cm⁻¹, which is the carboxylate symmetric stretching (C═O) peak. However, the peak at 1700 cm⁻¹ is obviously appeared when the solvent of the reaction system is methanol. It is shown that methanol is more suitable as a solvent than alcohol when Fe₃O₄NCs@SiO₂-EDTA nanoparticles are prepared.

### Embodiment 4. Preparation of Functional Peptide Modified Magnetic Composite Nanobead 4

What is different from Embodiment 1 is:

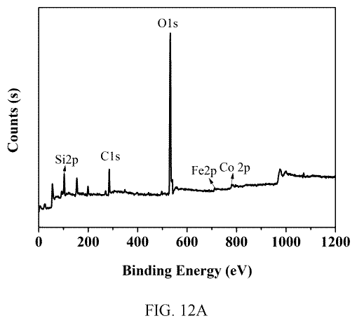

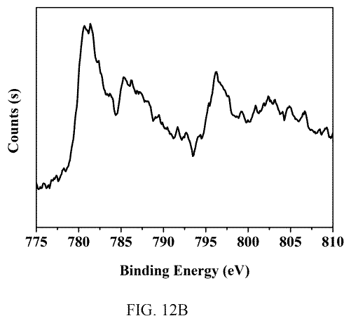

In preparation step (4) of functional peptide modified magnetic composite nanobeads, 4 ml of 1 mol/L CoCl₂ solution is added into the reaction mixture, and other conditions remain unchanged. The XPS full spectrum of the obtained magnetic composite nanobeads and the Co 2p photoemission spectrum are respectively shown in FIG. 12A and FIG. 12B. It may be seen that Co²⁺ ions are successfully chelated on the surface of Fe₃O₄NCs@SiO₂-EDTA.

### Embodiment 5. Preparation of Functional Peptide Modified Magnetic Composite Nanobead 5

In order to investigate the stability of magnetic beads prepared by different fluoresce labeled functional peptides, this embodiment is carried out. In this embodiment, the preparation process of magnetic composite nanobeads is the same as Embodiment 1, but a difference is that fluorescein used to label functional peptides is fluorescein isothiocyanate (FITC) and Rhodamine B, respectively.

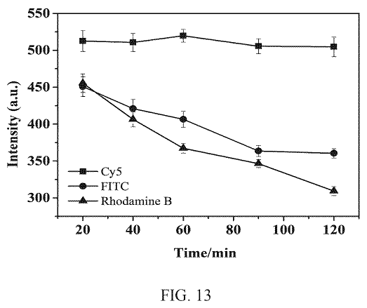

The result is shown in FIG. 13. It may be seen that the magnetic composite nanobeads prepared by Cy5 labeled functional peptide have best fluorescence stability in acid buffer system. As many literatures show that BACE1 has a strongest activity in acid buffer system, the functional peptide labeled with fluorescein Cy5 is suitable for preparing magnetic composite nanobeads to detect BACE1.

### Embodiment 6. Preparation of Functional Peptide Modified Magnetic Composite Nanobead 6

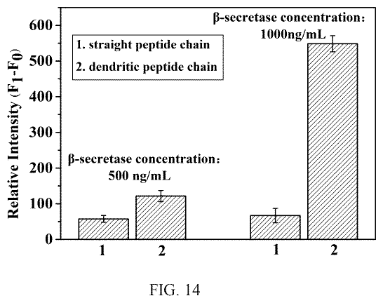

In this embodiment, the preparation process of magnetic composite nanobead is the same as the preparation process in Embodiment 1, the difference is that the amino acid sequence of the used functional peptide is: Cys-Leu-Gly-Gly-Glu-Val-Asn-Leu-Asp-Ala-Glu-Phe-Gly-Gly-Leu-His-His-His-His-His-His (SEQ ID No.3), which is inserted His-tag into a N-terminal of the peptide chains, namely a straight peptide chain. Compared with a dendritic peptide chain in Embodiment 1, the result of BACE1 detection is shown in FIG. 14 (No. 1 is a straight peptide chain and No. 2 is a dendritic peptide chain). It may be seen that the fluorescence intensity using the dendritic peptide chain modified magnetic composite nanobeads is higher than the fluorescence intensity using the straight peptide chain modified magnetic composite nanobeads, indicating the dendritic peptide chain is modified on the surface of the magnetic composite nanobeads, may better react with BACE1 and has more stable performance.

### Embodiment 7. Preparation of Functional Peptide Modified Magnetic Composite Nanobead 7

In this embodiment, the preparation process of magnetic composite nanobead is the same as the preparation process in Embodiment 1, the difference is that the amino acid sequence of the used functional peptide is: Cys-Gly-Gly-Ser-Ser-Glu-Val-Asn-Leu-Asp-Ala-Glu-Phe-Ser-Ser-Gly-Leu-His-His-His-His-His-His (SEQ ID No.1).

### Embodiment 8. Preparation of Functional Peptide Modified Magnetic Composite Nanobead 8

In this embodiment, the preparation process of magnetic composite nanobead is the same as the preparation process in Embodiment 1, the difference is that the amino acid sequence of the used functional peptide is: Lys-Thr-Glu-Glu-Ile-Ser-Glu-Val-Asn-Leu-Asp-Ala-Glu-Phe-Arg-His-Asp-Lys-Cys-His-His-His-His-His-His (SEQ ID No.2).

### Embodiment 9 Application of Functional Peptide Modified Magnetic Composite Nanobead 1

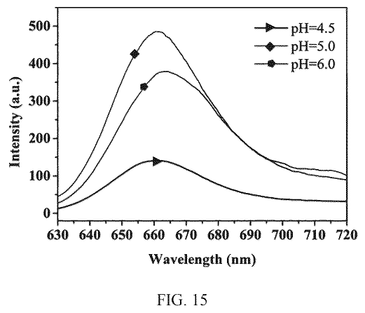

The purpose of this embodiment is to investigate the influence of different pH buffer on the detection of BACE1. Firstly, magnetic composite nanobeads prepared in Embodiment 1 are respectively dispersed in 20 mM HAc-NaAc buffer with different pH values of 4.5, 5.0 and 6.0, then, 0.1% triton and the same amount of BACE1 are added into the mixture. After that, the reaction mixture is incubated for 2 h at 37° C. Finally, the supernatant of the reaction mixture is collected by magnetic separation and its fluorescence intensity is monitored. The result of BACE1 detection is shown in FIG. 15, when the pH value of the HAc-NaAc buffer is 5.0, the detection performance of magnetic composite nanobeads is the best.

### Embodiment 10. Application of Functional Peptide Modified Magnetic Composite Nanobead 2

The purpose of this embodiment is to verify that the functional peptide modified magnetic composite nanobead prepared in Embodiment 1 may be used to detect BACE1, specific operation steps of the embodiment are as follows.

Firstly, functional peptide modified magnetic composite nanobeads are dispersed in HAc-NaAc buffer (pH=5.0, 0.1% triton), and then different amount of BACE1 are added into the mixture in a final reaction volume of 600.0 μL. Subsequently, the reaction mixture is incubated for 2 h at 37° C. Finally, the supernatant of the reaction mixture is collected by magnetic separation and its fluorescence intensity is monitored.

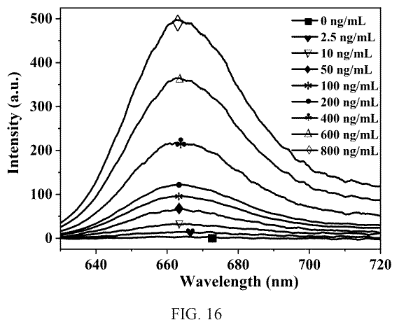

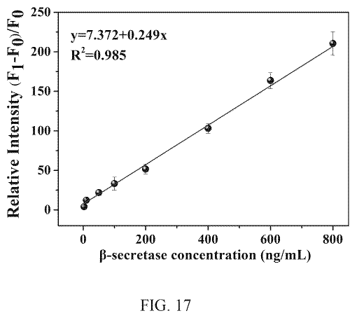

As shown in FIG. 16, a dramatic increase in the fluorescence intensity of the supernatant is observed with the increasing concentration of BACE1 (0 nanogram per milliliter (ng/mL), 2.5 ng/mL, 10 ng/mL, 50 ng/mL, 100 ng/mL, 200 ng/mL, 400 ng/mL, 600 ng/mL and 800 ng/mL), indicating a good response to the variation in BACE1 concentration. As shown in FIG. 17, there is a good linear relationship between the value of (I_{F}−I_{F0})/I_{F0} (where I_{F0} and I_{F} represent the fluorescence intensity of the supernatant in the absence and presence of BACE1, respectively.) and the BACE1 concentration in the range from 2.5 ng/mL to 800 ng/mL. The linear regression equation is ΔI_{F}=7.372+0.249c (ng/mL) (R²=0.985) and the limit of detection (LOD) is estimated to be 0.62 ng/mL.

### Embodiment 11. Detection of Anti-Interference Ability of BACE1 by Functional Peptide Modified Magnetic Composite Nanobead

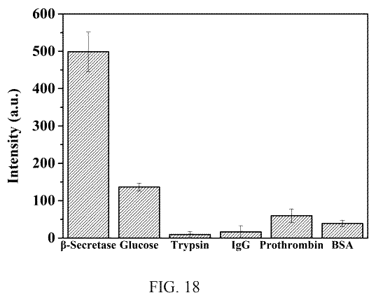

In this embodiment, the anti-interference ability of detecting BACE1 is investigated by using functional peptide modified magnetic composite nanobead, the detection steps are the same as Embodiment 10, but differences are as follows: the detection target is replaced by glucose oxidase, trypsin, human immunoglobulin G (IgG), prothrombin and bovine serum albumin (BSA) which may exist in an actual sample, respectively. As shown in FIG. 18, only BACE1 exhibits obviously change of fluorescence response, and other interferences have no obvious changes, indicating the functional peptide modified magnetic composite nanobead as nanoprobes has strong anti-interference ability and is suitable for the detection of BACE1 in complex environment.

### Embodiment 12. Feasibility of Screening Inhibitors with Functional Peptide Modified Magnetic Composite Nanobead

The purpose of this embodiment is to verify the potential application of the functional peptide modified magnetic composite nanobead in AD drug screening, specific operation steps of the embodiment are as follows.

Firstly, the different amount of commercial BACE1 inhibitors (BACE1 Inhibitor IV) are mixed with 800 ng/mL BACE1 in HAc-NaAc buffer (pH=5.0, 0.1% triton) and incubated at 37° C. for 10 min. Then 70 μL of 5 mg/mL magnetic composite nanobead solution and HAc-NaAc buffer are added into the mixture solution in a total reaction volume of 600.0 μL. Subsequently, the reaction mixture is incubated for 2 h at 37° C. Finally, the supernatant of the reaction mixture is collected by magnetic separation and its fluorescence intensity is monitored.

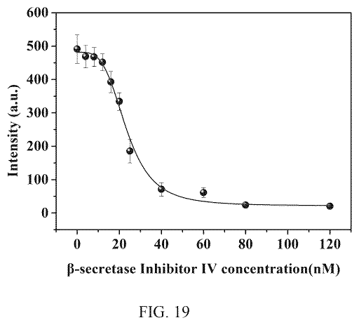

As shown in FIG. 19, when 800 ng/mL BACE1 is incubated with the different concentration of Inhibitor IV, the fluorescence intensity of the supernatant of the reaction mixture decreased with increasing the concentration of inhibitor I, indicating a gradual weakening of BACE1 activity. The inhibition efficiency of the inhibitor is calculated using the formula: (I_{F0′}−I_{Fi}/I_{F0})×100%, where I_{F0′} and I_{Fi} represent the fluorescence intensity of the detection system in the absence and presence of inhibitor IV, respectively. An inhibitory ability of inhibitor is described by IC₅₀, which is the inhibitor concentration required to reduce enzyme activity by 50%. The IC₅₀ value of inhibitor IV is calculated to be 20 nanomoles per liter (nmol/L), which is in agreement with those provided by BACE1 inhibitor suppliers. Above results show the functional peptide modified magnetic composite nanobead provided by the disclosure may be used for screening BACE1 inhibitors.

To sum up, the functional peptide modified magnetic composite nanobead provided by the disclosure has special microstructure and excellent properties, and its preparation method is mild and is beneficial to a large-scale preparation. More importantly, the functional peptide modified magnetic composite nanobead may be used for the detection of BACE1 with the advantages of high sensitivity, strong anti-interference ability and small background signal. The linear range of BACE1 detection is 2.5 ng/mL-800 ng/mL, and the detection limit is 0.62 ng/mL. The functional peptide modified magnetic composite nanobead is also used for screening BACE1 inhibitors, which has important application potential in an early diagnosis and treatment of AD and medicine development.

Finally, it should be explained that above is only optional embodiments of the present disclosure, and does not limit the present disclosure. Although the present disclosure has been described in detail with reference to the foregoing embodiments, it is still possible for a person skilled in the art to modify the technical scheme described in the foregoing embodiments, or to replace some technical features by equivalents. Any modification, equivalent replacement, improvement, etc. made within the spirit and principle of the present disclosure should be included in the scope of protection of the present disclosure.

What is claimed is:

1. A functional peptide modified magnetic composite nanobead, wherein a chemical composition of the magnetic composite nanobead is: ferroferric oxide nanoclusters used as magnetic cores, a surface of the magnetic cores coated with a silica shell, a surface of the silica shell connected with an ethylene diamine tetraacetic acid modification layer, Co²⁺ or Ni²⁺ metal ions chelated on the modification layer, and a Cy5 fluorescein labeled functional peptide coupled with the Co²⁺ or Ni²⁺ metal ions coated on a surface of the magnetic composite nanobead; and wherein an amino acid sequence of the Cy5 fluorescein labeled functional peptide is shown in any one of
(1) Cys-Gly-Gly-Ser-Ser-Glu-Val-Asn-Leu-Asp-Ala-
Glu-Phe-Ser-Ser-Gly-Leu-His-His-His-His-His-His;
(2) Lys-Thr-Glu-Glu-Ile-Ser-Glu-Val-Asn-Leu-Asp-
Ala-Glu-Phe-Arg-His-Asp-Lys-Cys-His-His-His-His-
His-His;
(3) Cys-Leu-Gly-Gly-Glu-Val-Asn-Leu-Asp-Ala-Glu-
Phe-Gly-Gly-Leu-His-His-His-His-His-His;
and
(4) [(His-His-His-His-His-His)4Lys]-
Gly-Gly-Pro-Pro-Pro-Pro-Pro-Pro-Pro-Pro-Pro-Gly-
Glu-Val-Asn-Leu-Asp-Ala-Glu-Phe-Asp-Asp-Lys-Gly-
Gly-Asp-Asp-Asp-NH₂.

2. The preparation method of the functional peptide modified magnetic composite nanobead according to claim 1, comprising following steps:
(1) preparing the ferroferric oxide nanoclusters by a solvothermal method;
(2) taking the ferroferric oxide nanoclusters prepared in the step (1) as the magnetic cores, and adopting an ultrasonic-assisted sol-gel method to prepare silica-coated ferroferric oxide nanocluster particles;
(3) dispersing the silica-coated ferroferric oxide nanocluster particles prepared in the step (2) in methanol, respectively adding glacial acetic acid and sodium N-(trimethoxysilyl propyl) ethylenediamine triacetate with a mass ratio of 1:2-1:10, carrying out a mixture reaction by ultra-sonication at 150-300 W for 1-3 h, and after the reaction is completed, washing the resulted materials with deionized water and acetone respectively to obtain ethylene diamine tetraacetic acid modified (EDTA-modified) silica-coated ferroferric oxide nanoclusters;
(4) ultrasonically dispersing the EDTA-modified silica-coated ferroferric oxide nanoclusters prepared in the step (3) in water, then adding aqueous solution of NiCl₂ or CoCl₂, wherein a mass ratio of the EDTA-modified silica-coated ferroferric oxide nanoclusters to the NiCl₂ or the CoCl₂ is 1:10-1:100, after the mixed solution reacts for 1.5-3 h, and then washing the resulted materials with the deionized water for three times to obtain Ni²⁺ or Co²⁺ chelated magnetic composite nanobeads; and
(5) dispersing the metal ions chelated magnetic composite nanobeads as-prepared in the step (4) in phosphate-buffered saline (PBS) buffer, then adding 5-15 μmol/L aqueous solution of functional peptide, incubating a reaction mixture at 37° C., magnetically separating and washing the resulted materials with the PBS buffer for 3-5 times until a fluorescence intensity of a washing solution tends to zero, and then obtaining a functional peptide modified magnetic composite nanobeads.

3. The preparation method according to claim 2, wherein specific steps of preparing the ferroferric oxide nanoclusters by the solvothermal method mentioned in the step (1) are as follows:
dissolving ferric chloride and sodium acetate in ethylene glycol, then adding sodium acetate and the deionized water to form mixed solution after being completely dissolved, wherein a mass ratio of the ferric chloride, the sodium acetate and the deionized water is 1:3:1, adding the mixed solution into a full-automatic high-temperature and high-pressure reaction kettle, setting a program, firstly heating to 140-170° C., keeping for 1.5-3 h, then heating to 180-220° C., keeping for 2-8 h, wherein a pressure in a whole reaction process is 0.01-0.1 Pa, and after the reaction is completed, taking out and washing a precipitate in the reaction kettle with the acetone and absolute ethanol respectively, and then obtaining the ferroferric oxide nanoclusters.

4. The preparation method according to claim 2, wherein specific steps of preparing the silica-coated ferroferric oxide nanocluster particles by the ultrasonic-assisted sol-gel method mentioned in the step (2) are as follows:
firstly, taking a certain amount of ferroferric oxide nanocluster particles, adding mixed solvent of the deionized water and the absolute ethanol with a volume ratio of 1:5-4:5, and uniformly dispersing by ultrasonic; then adding concentrated ammonia water and tetraethyl orthosilicate under an ice water bath environment, wherein a volume ratio of the tetraethyl orthosilicate to the concentrated ammonia water is 1:10-1:20, performing an ultrasonic reaction for 20-60 min in a power range of 180-360 W, and after the reaction is completed, washing twice with the absolute ethanol and the deionized water respectively, and then obtaining the silica-coated ferroferric oxide nanocluster particles.


SEQUENCE LISTING (4 sequences)

SEQ ID NO: 1 - protein, 23 aa, synthetic construct
CGGSSEVNLDAEFSSGLHHHHHH

SEQ ID NO: 2 - protein, 25 aa, synthetic construct
KTEEISEVNLDAEFRHDKCHHHHHH

SEQ ID NO: 3 - protein, 21 aa, synthetic construct
CLGGEVNLDAEFGGLHHHHHH

SEQ ID NO: 4 - protein, 30 aa, synthetic construct
XKGGPPPPPPPPPGEVNLDAEFDDKGGDDD